(19) United States
(12) Patent Application Publication
(10) Pub. No.: US 2022/0168495 A1
(43) Pub. Date: Jun. 2, 2022

(54) GARMENT INCLUDING A MICRO-PUMP FOR NON-FLUID MANAGEMENT TISSUE THERAPIES

(71) Applicant: KCI LICENSING, INC., San Antonio, TX (US)

(72) Inventor: Christopher Brian LOCKE, Bournemouth (GB)

(21) Appl. No.: 17/440,008

(22) PCT Filed: Mar. 24, 2020
(86) PCT No.: PCT/US2020/024416

§ 371 Date: Sep. 16, 2021

Related U.S. Application Data
(60) Provisional application No. 62/829,365, filed on Apr. 4, 2019.

(51) Int. Cl. A61M 1/00; A41D 1/00

(52) U.S. Cl. CPC A61M 1/962; A61M 1/964; A41D 1/002; A61M 2210/086; A61M 2205/3331; A61M 2205/3368; A61M 2207/00; A61M 2205/3324

(57) ABSTRACT

A garment includes a cover, a pump coupled to the cover, and a control system operably coupled to the pump. The cover is configured to surround a limb or a joint and to prevent air from entering or leaving an enclosed region between the limb or the joint. The pump is configured to remove air from the enclosed region. The control system is configured to control the pump and to regulate a negative pressure within the enclosed region. In some embodiments, the control system is configured to regulate a negative pressure of the enclosed region based on mobility data from a sensor.

### CROSS-REFERENCE TO RELATED APPLICATIONS

This application claims the benefit of priority to U.S. Provisional Application No. 62/829,365, filed on Apr. 4, 2019, which is incorporated herein by reference in its entirety.

### BACKGROUND

The present disclosure relates generally to tissue recovery products. More specifically, the present disclosure relates to the use of a garment that applies negative pressure to injured limbs and joints to improve recovery and healing time.

The application of negative pressure to wounds and damaged tissue has been shown to improve wound recovery times. Benefits of negative pressure therapies have also been observed in the treatment of injured limbs and joints. These benefits are of particular interest in the field of sports medicine, and as a therapy for athletes who desire to return to mobility and full function very quickly. Devices and methods for the effective delivery of negative pressure to injured limbs and joints is desired.

### SUMMARY OF THE INVENTION

One implementation of the present disclosure is a garment. The garment includes a cover configured to substantially surround a limb or a joint and sealably engage with the limb or the joint. The cover is configured to substantially prevent air from entering or leaving an enclosed region formed between the cover and the limb or joint. The garment includes a pump and a control system operably coupled thereto. The pump is coupled to the cover and configured to remove air form the enclosed region. The control system is configured to control the pump and to regulate a negative pressure within the enclosed region.

In some embodiments, the control system includes a sensor configured to measure mobility data. The control system may be configured to determine whether a user is moving or at rest based on the mobility data. The control system may be configured to maintain an increased negative pressure based on a determination that the user is at rest and to maintain a decreased negative pressure based on a determination that the user is moving. The garment may further include a valve operably coupled to the control system. The valve may be configured to allow air to enter the enclosed region. The control system may be configured to open the valve based on a determination that the user is moving and to close the valve based on a determination that a user is at rest.

In any of the above embodiments, the control system may be detachably coupled to at least one of the cover and the pump. In any of the above embodiments, the cover may be disposable and at least one of the pump and the control system may be reusable.

In any of the above embodiments, the control system may include a power source and an electro-mechanical pressure switch electrically coupled thereto. The electro-mechanical pressure switch may be configured to couple the pump to the power source in response to the pressure exceeding a threshold value. In any of the above embodiments, the control system may be configured to maintain the pressure within the enclosed region in a range between approximately negative 120 mm Hg and negative 145 mm Hg.

In any of the above embodiments, the control system may include a power monitoring system configured to measure an amount of current supplied to the pump. The power monitoring system may be configured to deactivate the pump based on a determination that the amount of current is below a threshold current value.

In any of the above embodiments, the garment may further include a sensor configured to collect data including at least one of mobility data and a condition of the enclosed region. The control system may further include a transceiver configured to transmit the data to a user device. The sensor may be one of a temperature sensor, a humidity sensor, a pressure sensor, and a pH sensor.

In any of the above embodiments, the garment may further include at least one of a filter configured to minimize odors from escaping the enclosed region and a filter configured to prevent ingress of fluids into the pump.

Another implementation of the present disclosure is a system. The system includes a power source configured to supply power to a pump, and a sensor electrically coupled to the power source and the pump. The system is configured to maintain an increased negative pressure within an enclosed region between a limb or a joint and a cover when a user is at rest and to maintain a decreased negative pressure within the enclosed region when the user is moving.

In some embodiments, the system includes a processing circuit operably coupled to the pump and the sensor. The processing circuit may be configured to determine whether the user is moving or at rest based on mobility data from the sensor. The processing circuit may be configured to maintain an increased negative pressure based on a determination that the user is at rest and to maintain a decreased negative pressure based on a determination that the user is moving.

In some embodiments, the system may be configured to maintain an increased negative pressure by at least one of activating the pump, increasing an operating speed of the pump, and closing a valve. The system may be configured to maintain a decreased negative pressure by at least one of deactivating the pump, reducing an operating speed of the pump, and opening a valve.

In some embodiments, the system includes memory configured to store a threshold current value. The system may also include a processing circuit operably coupled to the memory, the power source, and the pump. The processing circuit may be configured to monitor an amount of current supplied to the pump, and to deactivate the pump based on a determination that the amount of current is below the threshold value. In some embodiments, the memory is further configured to store a threshold rate of change and a cycling frequency for activating a deactivating the pump. The processing circuit may be configured to reduce the cycling frequency based on a determination that the rate of change is less than the threshold rate of change.

In some embodiments, the system further includes a user interface and a processing circuit operably coupled thereto. The processing circuit may be configured to generate an alert based on a determination that the processing circuit is separated from the pump. The user interface may be configured to display the alert.

In some embodiments, the system includes a locking member and a transceiver. The processing circuit may be configured to prevent removal of a processing circuit from the cover. The processing circuit may also be configured to operate the locking member in response to commands received by the transceiver.

Another implementation of the present disclosure is a method of making a garment. The method includes providing a cover configured to substantially surround and sealably engage at least one of a limb and a joint to form an enclosed region, providing a pump configured to draw a negative pressure within the enclosed region, and providing a control system configured to control the pump and regulate a negative pressure within the enclosed region. The method further includes integrating the pump into the cover. The method also includes coupling the control system to at least one of the cover and the pump and electrically coupling the pump to the control system.

In some embodiments, the method further includes providing a valve configured to allow air to enter the enclosed region and integrating the valve into the cover.

In some embodiments, the method further includes providing a sensor configured to activate the pump in response to the pressure exceeding a threshold value, and providing a power source. The method may include integrating the sensor into the cover and coupling the power source to the cover. The method may further include electrically coupling the sensor to the pump and the power source.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a front perspective view of a negative pressure therapy garment, according to an exemplary embodiment.

FIG. 2 is a front view of a control module and a pump module of a negative pressure therapy garment, according to an exemplary embodiment.

FIG. 3 is a side cross-sectional view of a pump module of a negative pressure therapy garment, according to an exemplary embodiment.

FIG. 4 is a schematic diagram of an electrical circuit of a negative pressure therapy garment, according to an exemplary embodiment.

FIG. 5 is an operational schematic of a negative pressure therapy garment, according to an exemplary embodiment.

FIG. 6 is a block diagram showing a method of making a negative pressure therapy garment, according to an exemplary embodiment.

### DETAILED DESCRIPTION

### Overview

Referring generally to the FIGURES, a garment for applying negative pressure to injured limbs and/or joints is provided, according to various exemplary embodiments. The garment includes a cover configured to seal off an enclosed region between the cover and the limb or joint, for example by sealably engaging with a user's skin or tissue. The garment includes a micro-pump configured to apply a negative pressure to the enclosed region. The pump is fluidly coupled to the enclosed region and also to an environment outside of the cover. The garment also includes a control system configured to control the pump to regulate a negative pressure within the enclosed region. The garment may be configured to coordinate the application of negative pressure with a user's movements, which can, advantageously, minimize user discomfort and improve user mobility.

The garment may be an occlusive limb cover that fully surrounds the limb or joint. The cover may include a hollow sleeve configured to receive the limb or joint. The pump may be integrated into an outer wall of the hollow sleeve. The pump may be a compact micro-pump in order to reduce operational noise.

The control system may include reusable electronic equipment including a power source. The control system may be detachably coupled to the cover so that it may be re-used with other devices. The control system may be configured to coordinate operation of the pump with user movement, for example, by utilizing a mobility sensor that can determine at least one of user orientation and degree of movement.

In some implementations, the control system may be configured to monitor pump operation and to modify control parameters to minimize power consumption. Feedback to the control system, based on pump operational information and sensor data, may also be utilized to maximize the effectiveness of the treatment. For example, the data may be transmitted to a user interface from which a user may monitor treatment progress. These and other features and advantages of the garment are described in detail below.

### Garment Construction

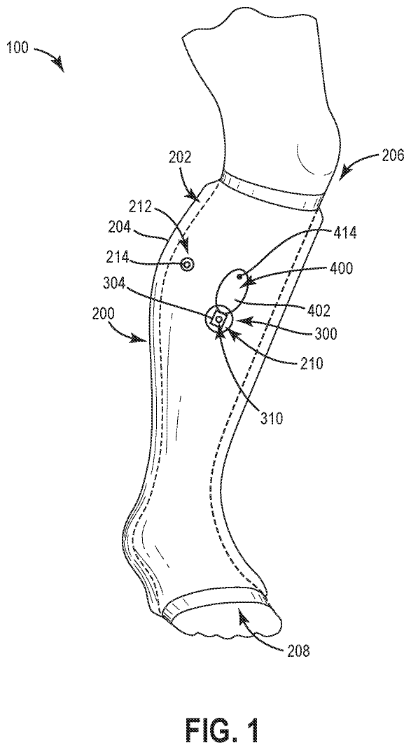

Referring now to FIG. 1, a garment 100 is shown, according to an exemplary embodiment. The garment 100 includes a cover 200 configured to receive a person's limb or joint. As shown in FIG. 1, the cover 200 is configured to receive a portion of a person's leg, including a lower portion of the leg and a foot. The cover 200 is configured to substantially surround the leg, forming an enclosed region 202 between the cover 200 and the leg. As shown in FIG. 1, the cover 200 is configured to sealably engage with the leg to prevent air from entering or leaving the enclosed region 202. In the embodiment of FIG. 1, an upper end of the cover 200 is configured to seal against a person's skin below the knee. A lower end of the cover 200 is configured to seal against the person's foot just above their toes.

As shown in FIG. 1, the garment 100 includes a pump module 300 and a control module 400 coupled thereto. The pump module 300 includes a pump 302 configured to remove air from the enclosed region 202. As shown in FIG. 1, the pump 302 is disposed in the cover 200, in an opening in a lower leg portion of the cover 200. As shown in FIG. 1, the pump 302 fluidly couples the enclosed region 202 to an environment surrounding the cover 200 (e.g., external to the cover 200, etc.).

In some implementations, the pump module 300 is configured to regulate a pressure of the enclosed region 202. According to an exemplary embodiment, the pump module 300 includes an electro-mechanical pressure switch operably coupled to the pump 302. The switch may be configured to complete an electrical connection to the pump 302 when the pressure within the enclosed region 202 exceeds a threshold value. In some embodiments, the pump module 300 includes additional sensors. The sensors may be configured to monitor conditions (e.g., temperature, pressure, humidity, etc.) in the enclosed region 202 or external to the cover 200. Alternatively, the sensors may be mobility sensors (e.g., accelerometers, etc.) configured to measure mobility data (e.g., angular orientation, degree of movement, etc.).

As shown in FIG. 1, the garment 100 includes a control module 400. The control module 400 is configured to control the pump 302 and to regulate a negative pressure within the enclosed region 202 between the cover 200 and the leg. As referred to herein, negative pressure refers to negative relative pressure referenced to atmospheric conditions, or reduced absolute pressure (e.g., a pressure less than 101.3 kPa absolute pressure, etc.).

The control module 400 includes electronic equipment including a power source and a pump driver or waveform driver. In some embodiments, the control module 400 include a processing circuit configured to receive and interpret sensor data. The processing circuit may be configured to determine whether a user is moving or at rest, a leak rate of air from the enclosed region 202, heath/diagnostic data from the pump or sensors, and/or other processing functions. The processing circuit may be configured to control the pump based on sensor data to optimize the performance of the garment 100.

In some embodiments, the processing circuit may be configured to coordinate the application of negative pressure with user movement. For example, the processing circuit may be configured to maintain an increased negative pressure based on a determination that the user is at rest and/or to maintain a decreased negative pressure based on a determination that the user is moving. Among other benefits, coordinating the application of negative pressure with movement improves mobility and reduces user discomfort.

According to an exemplary embodiment, the garment 100 includes a power monitoring system configured to measure the current drain from the power source and to determine when the pump is operational and/or when steady-state conditions have been achieved in the enclosed region 202. In some implementations, the power monitoring system includes the processing circuit. The power monitoring system may be configured to periodically activate the pump in order to maintain a negative pressure within a suitable range. The power monitoring system may include an ammeter configured to measure current drain on the power source while the pump is operational. The current data may be utilized to determine a leak rate of air from the enclosed region. The power monitoring system may be configured to control the frequency of pump operation in response to the leak rate to minimize pump operation and overall power consumption.

The control module 400 may include a user interface configured to receive and display sensor data, an operating status of the garment 100, or alerts/notifications generated by the processing circuit. According to an exemplary embodiment, the control module 400 is communicatively coupled to a user device (e.g., a smart device, a mobile phone, a tablet, a laptop, or another remote computing device). The control module 400 may be configured to transmit sensor data to the user device so that a user may monitor treatment progress. The sensor data may be monitored and manipulated from an application on the user device. In some implementations, the control module 400 may be configured to transmit notifications and alerts to the user device (e.g., notifying the user of a malfunction with the device, a sudden loss of negative pressure, etc.). Additionally, the control module 400 may be configured to receive pump operating commands from the user device and/or information about a user's activities (e.g., whether the user is at rest or moving, etc.). Among other benefits, interactive control and monitoring of the garment 100 may be used to assist with future healing cycles of repetitive injuries to limbs or joints.

In the embodiment of FIG. 1, the control module 400 is detachably coupled to the cover 200. The control module 400 is detachably coupled to a cover-mounted connector 304 of the pump module 300. Among other benefits, using removable components reduces replacement costs for the garment 100, as the control module 400 may be replaced separately from the other components.

### Cover

An exemplary embodiment of a cover 200 for the garment 100 is shown in FIG. 1. The cover 200 includes an outer wall 204 defining a hollow sleeve. The cover 200 is configured to receive a person's limb or joint such that it substantially surrounds the limb or joint. In the embodiment of FIG. 1, the cover 200 is configured to receive a lower leg portion and a foot portion of a person's leg. In other embodiments, the cover 200 may be configured to receive a person's arm. In yet other embodiments, the cover 200 may be configured to receive a swollen joint such as a knee or elbow.

According to an exemplary embodiment, the cover 200 is configured to sealably engage with a person's limb or joint to prevent air from entering or leaving the enclosed region 202. As shown in FIG. 1, a first end 206 (e.g., upper end) of the cover 200 is engaged with a person's skin, just below their knee. The first end 206 includes a cuff that is engaged with the skin. The cuff circumferentially surrounds the leg to form an air-tight seal between the enclosed region 202 and the surrounding environment. In some embodiments, the cuff includes a long or short stretch material that maintains compression between the cuff and the skin. As shown in FIG. 1, a second end 208 (e.g., lower end) of the cover 200 is engaged with the skin just above a person's toes. In the embodiment of FIG. 1, the second end 208 of the cover 200 also includes a cuff. In alternative embodiments, the second end 208 encloses an end of a person's foot.

The cover 200 may include a variety of compressive/expansive materials including plastics such as polyvinyl chloride and other materials. According to an exemplary embodiment, the cover 200 includes a material with low gas permeability (e.g., low gas transmission rates, etc.) to ensure an air-tight seal between the cover 200 and the leg. In some implementations, the cover 200 may include an occlusive dressing. The cover 200 may include a waxy coating and/or silicon adhesive to improve sealing between the enclosed region 202 and the surrounding environment. In some implementations, the cover 200 also includes an inexpensive wound pad, within the enclosed region, along an inner surface of the cover, to absorb moisture from the skin. According to an exemplary embodiment, the cover 200 is configured to be disposed of after use.

As shown in FIG. 1, a central portion of the cover 200, between the first end 206 and the second end 208 is loose fitting around the leg both for user comfort and to ensure that trapped air along the length of the leg can be transported easily to the pump. The cover 200 fits snugly around the leg when operational and may be easily hidden beneath a user's clothing, if desired, to conceal the device.

The cover 200 is configured receive pneumatic components of the garment 100. As shown in FIG. 1, the cover 200 includes a first opening 210 configured to receive the pump 302, a pressure switch, and the cover-mounted connector 304. The cover 200 also includes a second opening 212 configured to receive a valve 214. The valve may be one, or a combination of, an over-pressure relief valve (e.g., a mechanical pop-off valve), a manually actuated pressure release valve, or another type of valve. The cover 200 may include additional or fewer openings in various alternative embodiments.

In some embodiments, the cover 200 includes one or more connectors (e.g., electrical connectors) configured to couple (e.g., electrically connect) the electrical equipment (e.g., the pump, one or more sensors, etc.) to the cover 200 and/or to position the electrical equipment within the cover 200. The connectors may be one, or a combination of, of a variety of different connectors known to those of ordinary skill in the art.

### Pump Module

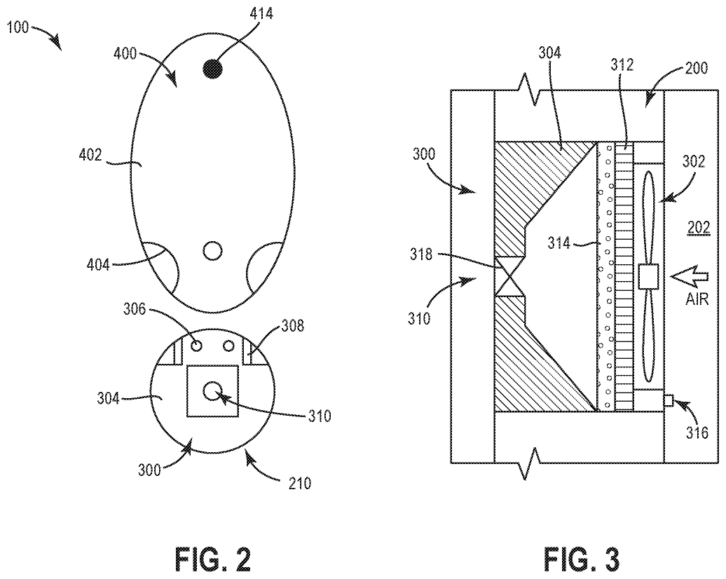

Referring now to FIGS. 1-4, a pump module 300 is shown, according to an exemplary embodiment. As shown in FIGS. 1-4, the pump module 300 includes a pump 302 and a cover-mounted connector, shown as connector 304. As shown in FIG. 2, the connector 304 is configured to operably couple the control module 400 to the pump module 300.

As shown in FIGS. 1-2, the connector 304 is coupled to the cover 200. In the exemplary embodiment of FIG. 1, the connector 304 is disposed within the first opening 210 of the cover 200 along an upper portion of the leg such that the pump module 300 may be easily accessed without limiting user mobility. As shown in FIGS. 1-2, the connector 304 is sealably coupled to the first opening 210 along a perimeter of the cover-mounted connector 304. The connector 304 may be coupled to the cover 200 using an adhesive product such as a silicon adhesive or another air-tight adhesive. In some embodiments, the cover 200 may be bonded directly (e.g., heat bonded) to the connector 304.

As shown in FIG. 2, the connector 304 includes a pair of leads 306 (e.g., electrical leads, terminals, etc.) configured to electrically couple the control module 400 to the pump module 300. According to an exemplary embodiment, the leads 306 are configured to power the pump. The leads 306 may also be configured to power one or more sensors that are included as part of the pump module 300. As shown in FIG. 2, the connector 304 also includes a plurality of mechanical latching points 308 configured to detachably couple the control module 400 to the pump module 300. The mechanical latching points 308 may include clips, tabs, or another form of mechanical connector. The mechanical latching points 308 may be configured to engage with a pair of sprung connectors or another mating connector on the control module 400. In other embodiments, the mechanical latching points 308 may include another form of detachable mechanical connector.

The pump 302 is configured to remove air from the enclosed region 202 (e.g., to transport air from the enclosed region 202, between the cover 200 and the leg (see also FIG. 1), to the surroundings, etc.). According to an exemplary embodiment, the pump 302 is disposable. A variety of low cost, quiet, and compact air pumps may be incorporated into the garment 100. According to an exemplary embodiment, the pump 302 is a micropump or microblower such as a Murata air pump.

As shown in FIG. 3, the pump 302 is coupled to and contained substantially within the connector 304. According to an exemplary embodiment, the pump 302 is coupled to the connector 304 along an inner surface of the connector 304. The pump 302 may be bonded, glued, or otherwise affixed to the inner surface of the connector 304. An outer surface of the connector 304, opposite the inner surface, is coupled to the cover 200. As shown in FIG. 3, the pump 302 is disposed proximate to a first end of the connector 304, adjacent to the enclosed region 202. An exhaust port 310 is centrally disposed at a second end of the connector 304. The size and shape of the connector 304 may be different in various alternative embodiments.

As shown in FIG. 3, the pump module 300 includes two filters, a charcoal filter 312 configured to minimize odors escaping from the enclosed region 202, and a hydrophobic filter 314 configured to prevent fluid ingress from the surroundings into the pump 302 and the enclosed region 202. In other embodiments, the number and/or arrangement of filters within the connector 304 may be different. As shown in FIG. 3, both the charcoal filter 312 and the hydrophobic filter 314 are disposed within the connector 304, downstream of the pump 302, between the pump 302 and an exhaust port of the connector 304. According to an exemplary embodiment, the hydrophobic filter 314 is disposed proximate to the second end of the connector 304 which may, advantageously, prevent fluid ingress through the exhaust port 310 to both the charcoal filter 312 and the pump 302.

As shown in FIG. 3, the pump module 300 includes a valve 318 disposed proximate to the second end of the connector 304, between the hydrophobic filter 314 and the exhaust port 310. In some embodiments, the valve 318 is a one-way check valve to prevent air from leaking into the enclosed region 202 when the pump 302 is non-operational. Alternatively, the valve 318 may be a solenoid valve operably coupled to the control module 400. In yet other embodiments, the valve 318 may include a manual control button disposed on an outer surface of the connector. The control button may include a spring that biases the button into a closed position to prevent inadvertent loss of negative pressure. The button may provide a functionality by which a user may decrease the negative pressure in the enclosed region 202 (e.g., increase the absolute pressure) to improve user comfort during periods of mobility.

The garment 100 is configured to maintain a negative pressure within the enclosed region 202. As shown in FIG. 3, the pump module 300 includes a sensor 316 coupled to the connector 304 and extending at least partially into the enclosed region 202. The sensor 316 is configured to measure a condition of the enclosed region 202. The sensor 316 may be one of a temperature sensor configured to measure a temperature of the enclosed region, a humidity sensor configured to measure a moister level of the enclosed region, a mobility sensor such as an accelerometer configured to measure user movement or a user's orientation, a pH sensor configured to measure a pH of a user's skin, or another type of sensor.

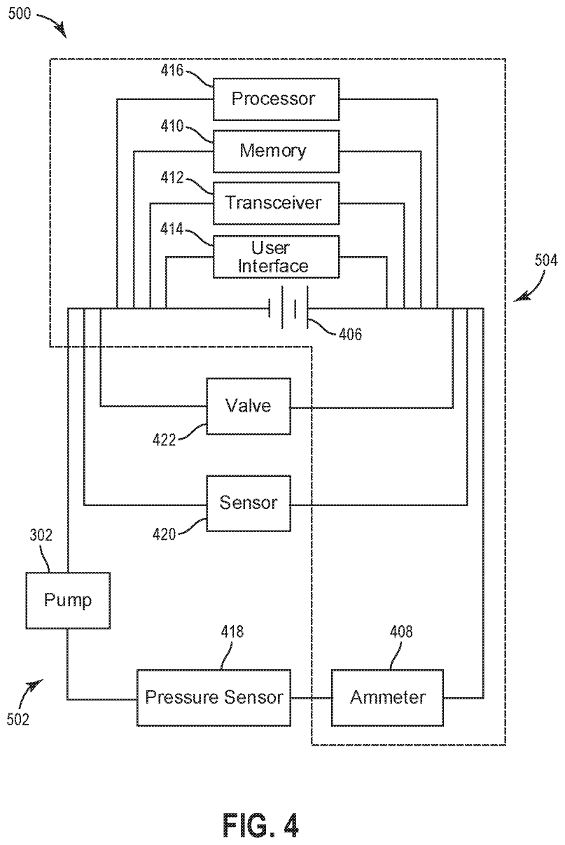

According to an exemplary embodiment, the sensor 316 is a pressure sensor configured to measure a pressure of the enclosed region 202. In the embodiment of FIG. 3, the sensor 316 includes an electro-mechanical pressure switch operably coupled (e.g., electrically connected to) to the pump 302, in series between the pump 302 and a power source (see also FIG. 4). The electro-mechanical pressure switch is configured to electrically couple the pump to a power source in response to the pressure exceeding a threshold value. The electro-mechanical switch may be biased by a spring into a closed position, so as to complete the electrical circuit between the pump 302 and the power source, when a pressure in the enclosed region 202 exceeds a threshold value. The threshold value may be determined based on a known therapeutic value of pressure or a range of pressures.

According to an exemplary embodiment, the electro-mechanical switch is configured to maintain a pressure within the enclosed region of approximately negative 125 mm Hg (e.g., −16.7 kPa relative pressure, 84.7 kPa absolute pressure), in a range between approximately negative 105 mm Hg and negative 145 mm Hg (e.g., a threshold value of approximately negative 105 mm Hg), or another suitable range of pressures based on the type of injury and its severity. In some implementations, the switch may further include an absorber component (e.g., a closed cell foam, padding, or another absorber) in order to dampen hysteresis and prevent sensor “flutter,” or to prevent the switch from alternating rapidly between an open and closed position when the pressure is approximately equal to the threshold value.

### Control Module

According to an exemplary embodiment, the garment 100 includes a control system configured to control the pump 302 and to regulate a negative pressure within the enclosed region 202. As shown in FIGS. 1-2, the control system includes a control module 400. The control module 400 includes a housing 402 configured to detachably couple the control module 400 to the pump module 300. The control module 400 includes a plurality of reusable electronic equipment for the garment 100. The equipment is contained substantially within the housing 402, which prevents water damage and provides an improved overall aesthetic appearance.

As shown in FIGS. 1-2, the housing 402 includes sprung connectors 404 that engage with the mechanical latching points 308 on the cover-mounted connector 304. The sprung connectors 404 may include metal clips, latches, or another form of mechanical connector. In some embodiments, the sprung connectors 404 also function as electrical connectors configured to engage with the leads 306 on the cover-mounted connector 304.

In some embodiments, the control module 400 is configured to identify whether the control module 400 is correctly connected to the pump module 300 (e.g., that the control module 400 is properly aligned with the pump module 300, that the control module 400 has fully engaged with the mechanical latching points 308, that an electrical connection has been established between the pump module 300 and the control module 400, etc.). The control module 400 may include a read switch or magnetic sensor structured to trigger an alarm if the control module 400 and the pump module 300 are misaligned. For example, the control module 400 may include a magnetic sensor integrated centrally between the sprung connectors 404. The pump module 300 may include an opposing magnet integrated into the cover-mounted connector 304. In some implementations, the opposing magnet may be integrated into the connector 304 proximate to the mechanical latching points 308. In the event the magnetic sensor isn't fully aligned with the magnet (e.g., in the event the control module 400 is detached from the pump module 300, etc.), the control module 400 may be configured to generate a notification alerting a user of misalignment. The notification may be an audible alarm, a visual notification (e.g., a light), a notification on a user's phone or smart device, or another suitable notification.

In some embodiments, the garment 100 is configured to prevent unauthorized or unintentional removal of the control module 400 from the pump module 300. For example, the connector 304 may include a locking member including a solenoid latch that prevents separation of the control module 400 from the connector 304 until a release command is received from a user device. The release command may be generated by entering a personal identification number or password into an application on the user device. Different controllable locking mechanisms may be utilized in various alternative embodiments. In some embodiments, the garment 100 includes an ultraviolet (UV) switching adhesive system to prevent unauthorized separation of the control module 400 from the pump module 300. The control module 400 may include a UV switching adhesive disposed proximate to the sprung connectors 404. The UV switching adhesive may be configured to adhere to the cover-mounted connector 304 in the absence of a light source. The pump module 300 may include an emitter (e.g., a UV light source, etc.) disposed on the cover-mounted connector 304 and configured to release the adhesive from the cover-mounted connector 304 upon receipt of the release command from the user device.

Referring now to FIG. 4, a schematic diagram of a circuit 500 for the garment 100 is shown, according to an exemplary embodiment. The garment 100 includes a plurality of electrical components configured to control the pump 302 and regulate a negative pressure within the enclosed region 202. In alternative embodiments, the control module 400 may include additional, fewer, and/or different components. As shown in FIG. 4, the circuit 500 is subdivided into two portions, a first portion 502 including electrical components for the pump module 300, and a second portion 504 including electrical components for the control module 400. In alternative embodiments, the position of electrical components within the circuit 500 may be different.

As shown in FIG. 4, the control module 400 includes a power source 406, an ammeter 408, memory 410, a transceiver 412, a user interface 414, and a processor 416. The power source 406 may include a battery such as a lithium-ion battery, or another compact or lightweight battery type. The power source 406 may be rechargeable. In some embodiments, the power source 406 may be recharged by separating (e.g., detaching, removing, etc.) the control module 400 from the pump module 300 and placing the control module 400 on a recharging station or otherwise coupling the control module 400 to a wall outlet. In other embodiments, the power source 406 may be removably coupled to the control module 400.

As shown in FIG. 4, the power source 406 is coupled (e.g., electrically coupled) to the pump 302 and a pressure sensor 418 in a series circuit arrangement. The pressure sensor 418 may be configured to operate the pump 302 substantially independently from the control module 400. According to an exemplary embodiment, the pressure sensor 418 is an electro-mechanical pressure switch whose position is determined based on the pressure in the enclosed region 202 (see also FIG. 1), as was described with reference to sensor 316 in FIG. 3. In other embodiments, the pressure sensor 418 may be a transducer configured to measure the pressure (e.g., the negative pressure relative to atmospheric pressure, etc.) in the enclosed region 202.

As shown in FIG. 4, the control module 400 is operably coupled to a second sensor, shown as sensor 418. The sensor 420 may be configured similar to sensor 316. The sensor 420 may be coupled to the cover-mounted connector 304 and extend at least partially into the enclosed region 202 so as to measure a condition of the enclosed region 202. According to an exemplary embodiment, the sensor 420 is configured to provide information related to a user's mobility (e.g., to measure mobility data such as a user's orientation, degree of movement, etc.). In some embodiments, the sensor 420 includes an accelerometer configured to measure the force and frequency of a user's movements (e.g., each step taken by a user, contact between a user's foot and a ground surface, or another force associated with user movement). In other embodiments, the sensor 420 includes a heart rate sensor or another health monitoring sensor, which could determine user movement based on increased heart rate, body temperature, skin moisture (e.g., perspiration), and other factors.

As shown in FIG. 4, the control module 400 is operably coupled to a valve 422. The valve 422 may be the same as valve 214 described with reference to FIG. 1 or valve 318 described with reference to FIG. 2. According to an exemplary embodiment, the valve 422 is a solenoid valve configured to allow air to enter the enclosed region 202 (see also FIG. 1) in response to a control signal generated by the control module 400. The control module 400 may be configured to open the valve 422 based on a determination that the user is moving in order to reduce pain and discomfort, or in response to a command from a user device indicating that the user is at rest (e.g., that the user is immobile, etc.).

The control module 400 includes a power monitoring system. The power monitoring system is configured to monitor and optimize pump 302 operation. The power monitoring system includes an ammeter 408 configured to measure an amount of current provided to the pump 302 by the power source 406. The ammeter 408 may include one of a variety of commercial current measurement devices known to those of ordinary skill in the art. As shown in FIG. 4, the ammeter 408 is integrated in a series circuit arrangement between the power source 406 and the pump 302. In other embodiments, the location of the ammeter 408 within the circuit 500 may be different.

Memory 410 for the control module 400 may be configured to store operating instructions for the garment 100. Memory 410 may also be configured to store control parameters. The control parameters may include a threshold value of pressure for the enclosed region 202. The threshold value of pressure may be a therapeutic pressure or range of pressures shown to facilitate healing or wound recovery. The threshold value of pressure may vary based on the type of injury, progress of treatment, and other factors. According to an exemplary embodiment, the control parameters include threshold values for the power management system. For example, the control parameters may include threshold values of current supplied to the pump 302 and below which the pump 302 should be deactivated. The control parameters may additionally include a cycling frequency for the pump 302 and a threshold rate of change of current between cycles.

The transceiver 412 may include a transmitter for transmitting information and/or a receiver for receiving information. According to an illustrative embodiment, the transceiver 412 is configured to communicate wirelessly with a user device (e.g., via Wi-Fi, Bluetooth, or another suitable wireless communication protocol). The user device may include a remote computing device such as a smart watch, a mobile phone, a laptop computer, a tablet, an internet of things (IoT) device, or another internet or network connected device. The transceiver 412 may be configured to transmit sensor data from at least one of the sensors 316, 420 to the user device. The sensor data may include at least one of temperature data, humidity data, pressure data, mobility data, and pH data. The sensor data may be accessed through an application on the user device. The application may be configured to provide guidance or a treatment regimen to a user of the garment 100 in order to maximize the effectiveness of the treatment.

According to an exemplary embodiment, the application is configured to tailor (e.g., adjust, modify, etc.) the treatment regimen based on sensor data. Sensor data provided to the user device throughout a healing cycle may also be utilized to optimize future healing cycles of repetitive injuries to the same limb, joint, or muscle group. For example, the user or the control system may identify a progression of movement (e.g., a rate of increase in user mobility over the treatment duration) that is optimal for recovery by comparing improvements in pain, wound appearance, heat measurements of tissues, and measured parameters with increases in the rate of mobility over the treatment period. Moreover, the application may be configured to share treatment information (e.g., through the cloud or between user devices) with others having similar injuries. The application may allow the user to compare healing times and rest-exercise regimens in order to further optimize the therapeutic benefits of the treatment (e.g., so that a user may learn and adapt their treatment style, so that the application may adapt its prescribed treatment regimen, etc.).

The transceiver 412 may also be configured to transmit notifications to the user device. For example, the transceiver 412 may be configured to transmit a notification to the user device alerting the user that they should rest to reduce the risk of further injury. The transceiver 412 may also be configured to transmit diagnostic data from one or more sensors 316, 420 to the user device. The diagnostic data may be health monitoring data for one or more sensors 316, 420, notification of a poor connection between the control module 400 and the pump module 300, notification of an operational or performance issue (e.g., issues with achieving a desired negative pressure within the enclosed region 202 (see also FIG. 1), etc.). The notification may be a text message or an application pop-up on the user device. Alternatively, the notification may be an audible or visual alert generated by the user interface 414.

According to an exemplary embodiment, the user interface 414 is configured to generate and display notifications and alerts. As shown in FIGS. 1-2, the user interface 414 includes an indicator 424 configured to report a condition of the enclosed region 202 or an operating condition or status of the garment 100. As shown in FIGS. 1-2, the indicator 424 includes a light emitting diode (LED) disposed on an outer surface of the housing 402. According to an exemplary embodiment, the indicator 424 is configured to provide a visual indication of an operating status to a user. The operating status may include remaining battery life, an operating status of the pump 302, an indication of alignment between the control module 400 and the pump module 300, etc. In other embodiments, the indicator 424 may include a speaker, an LED display, or another type of indicator known to those of ordinary skill in the art.

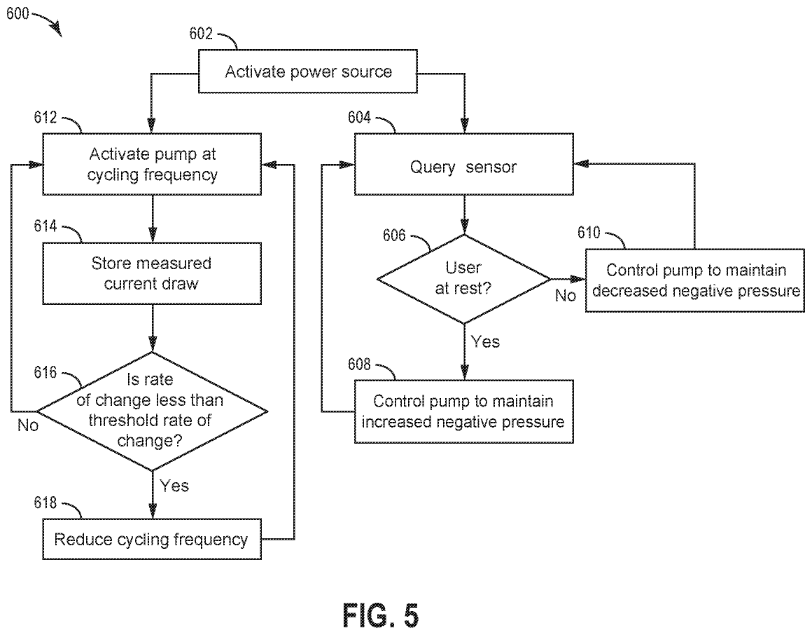

As shown in FIG. 4, the control module 400 includes a processing circuit, shown as processor 416. The processor 416 may be operably coupled each of the components in the control module 400 and configured to control interaction between the components. According to an exemplary embodiment, the processor 416 is configured to receive and interpret mobility data from the sensor 420. In some embodiments, the processor 416 may be configured to generate a control signal for at least one of the pump 302 and the valve 422 based on the mobility data from the sensor 420. The processor 416 may form part of the power management system and may be configured to control the pump 302 to minimize power consumption. The function of the processor 416 will be described in further detail with reference to FIG. 5.

### Pump Operation

Referring now to FIG. 5, a method 600 of operating the pump 302 (see also FIG. 1) is shown, according to an exemplary embodiment. The method 600 includes activating the power source 602 for the garment 100. The power source 406 may be activated by connecting the control module 400 to the pump module 300 or by actuating an on/off switch for the garment 100 after the control module 400 and the pump module 300 have been connected (e.g., aligned or otherwise connected).

The control module 400 is configured to coordinate the application of negative pressure to the enclosed region 202 with a user's movements. More specifically, the control module 400 is configured to maintain an increased negative pressure within the enclosed region 202 when the user is at rest and to maintain a decreased negative pressure within the enclosed region 202 when the user is moving. As shown in FIG. 5, the method 600 includes using the sensor 420 to control operation of the pump 302. The method 600 includes querying the sensor 604 to determine if the user is at rest 606. The sensor 420 may be configured to output sensor data indicative of user movement (e.g., a pulse, a voltage, etc.). The processor 416 may be configured to receive the sensor data and to identify a period of time (e.g., by querying a timer) between user movements. The processor 416 may be configured to compare the period of time with a threshold period of time stored in memory 410. Alternatively, the processor 416 may be configured to identify that the user is at rest based on a command received from the user device.

As shown in FIG. 5, the method 600 includes controlling the pump 302 to maintain an increased negative pressure 608 in the enclosed region 202 based on a determination that the user is at rest. According to an exemplary embodiment, the processor 416 is configured to generate a control signal that causes the pump 302 (e.g., to a pump driver, waveform driver, etc.) to increase the negative pressure in the enclosed region 202 (e.g., to decrease the absolute pressure in the enclosed region). The processor 416 may maintain an increased negative pressure by at least one of activating the pump 302, increasing an operating speed of the pump 302, and closing a valve 318, 422.

The method 600 further includes controlling the pump 302 to decrease and maintain a decreased negative pressure 610 in the enclosed region 202 based on a determination that the user is moving. According to an exemplary embodiment, the processor 416 is configured to generate a control signal that causes the pump 302 to decrease the negative pressure in the enclosed region 202 (e.g., to increase the absolute pressure in the enclosed region 202). The processor 416 may maintain the decreased negative pressure by at least one of deactivating the pump 302, reducing an operating speed of the pump 302, and opening a valve 422. In some implementations, the garment 100 may be configured to allow the pressure to decay naturally through patient movement and application leak to a lower pressure (e.g., −50 mm Hg or another suitable pressure) in order to reduce discomfort during periods of ambulation. The control module 400 may be configured to continuously query the sensor 420 to determine changes in the user's mobility. Alternatively, the control module 400 may be configured to reassert negative pressure to the enclosed region 202 after a given period of time has elapsed.

The method 600 includes controlling the pump 302 to regulate the pressure of the enclosed region 202 (see also FIG. 1) and to reduce power consumption. As shown in FIG. 5, the method 600 includes activating the pump 612. According to an exemplary embodiment, the processor 416 is configured to activate and deactivate the pump 302 at a first cycling frequency stored in memory 410. For example, the processor 416 may be configured to pole (e.g., to activate the pump 302, increase the operating speed of the pump 302, etc.) every 3 min., 6 min., or another suitable cycling frequency. The cycling frequency may vary depending on injury type, pressure requirements, and/or progression of treatment.

The method 600 may include monitoring the current drain during pump 302 operation (e.g., at the cycling frequency) using the power monitoring system. According to an exemplary embodiment, the processor 416 is configured to continue operating the pump 302 until the amount of current is below a threshold current value. More specifically, the processor 416 is configured to continue operating the pump 302 until at least one of two conditions have been achieved. A first condition includes operating the pump 302 continuously until the measured current drain (e.g., the current measured using ammeter 408) is less than or equal to approximately 80% or another fraction of the full-load operating current. A second condition includes operating the pump 302 continuously until the measured current drain is less than or equal to approximately 80% of the close-coupled current draw of the pump 302 (e.g., the anticipated close-coupled or full load current draw). A current draw below the threshold current indicates that steady-state operating conditions have been achieved in the enclosed region 202 (e.g., a largest negative pressure in the enclosed region 202 has been achieved, etc.). The threshold current value may be different in various alternative embodiments.

As shown in FIG. 5, the method 600 includes storing measured current data 614 from the power source 406 (e.g., the measured current drain during periods when the pump 302 is operational). According to an exemplary embodiment, the processor 416 is configured to receive and store data from the ammeter 408. The processor 416 may be configured to determine a rate of change of current during a single operating cycle of the pump 302 or between adjacent operating cycles (e.g., at the cycling frequency of the pump 302, etc.). As shown in FIG. 5, the method 600 includes comparing the measured rate of change of current with a threshold rate of change. The method 600 includes reducing the cycling frequency 618, from the first cycling frequency to a second cycling frequency, based on a determination that the measured rate of change is less than the threshold rate of change 616 (e.g., that the pump 302 does not need to be operated as frequently in order to maintain the required pressure in the enclosed region 202). Among other benefits, this control approach minimizes power consumption over the treatment duration.

The operations of method 600 are provided for illustrative purposes only and should not be considered limiting. Many alternatives are possible without departing from the inventive concepts disclosed herein. For example, the method may further include quantifying the leak rate from the cover. The leak rate may be quantified using current measurements from the ammeter 408, or by examining pressure measurements over time from a pressure transducer. Among other benefits, using a pressure transducer would allow for a more accurate calculation of leak rate of air into the enclosed region 202 (see also FIG. 1).

### Making a Garment for Negative Pressure Therapy

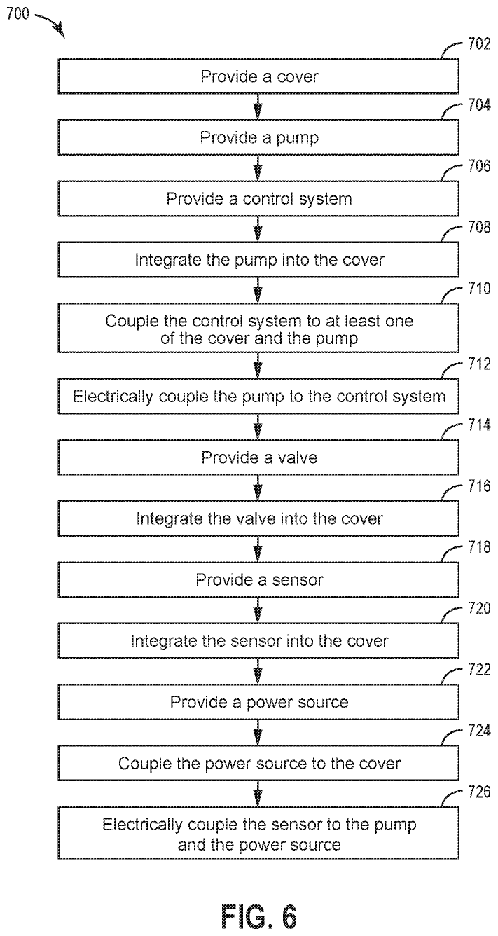

Referring now to FIG. 6, a method 700 of making a garment for negative pressure therapy is shown, according to an exemplary embodiment. In other exemplary embodiments, additional, fewer, and/or different operations may be performed. The method 700 includes providing a cover 702, providing a pump 704, and providing a control system 706. As described with reference to FIGS. 1-2, the control system 706 includes a control module 400. As shown in FIG. 6, the method 700 includes integrating the pump into the cover 708. In some embodiments, the pump may be integrated as part of a pump module into the cover. According to an exemplary embodiment, the components of the pump module are made from inexpensive materials to reduce the cost associated with damaging the cover or any cover-mounted component.

As shown in FIG. 6, the method 700 additionally includes coupling the control system to at least one of the cover and the pump 710. The method 700 further includes electrically coupling the pump to the control system 712. According to an exemplary embodiment, the control module 400 is detachably coupled (e.g., removably coupled) to the pump module such that the control module 400 may be reused with different covers.

The method 700 further includes providing additional electrical components that facilitate control and operation of the garment. Operations include providing a valve 714 (e.g., a solenoid valve or a manual discharge valve), a sensor 718 (e.g., an electro-mechanical pressure switch, etc.), and a power source 722 (e.g., a battery). The method 700 includes integrating the valve 716 and the sensor 720 into the cover. The method 700 includes coupling the power source to the cover 724, and electrically coupling the sensor to both the pump and the power source 726.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

What is claimed is:

1. A garment, comprising:
a cover configured to substantially surround a limb or a joint, and forming an enclosed region, wherein the cover is configured to engage with the limb or the joint to substantially prevent air from entering or leaving the enclosed region;
a pump coupled to the cover and configured to remove air from the enclosed region; and
a control system operably coupled to the pump, the control system configured to control the pump and regulate a negative pressure within the enclosed region.

2. The garment of claim 1, wherein the control system comprises a sensor configured to measure mobility data, wherein the control system is configured to determine whether a user is moving or at rest based on the mobility data, wherein the control system is configured to maintain an increased negative pressure based on a determination that the user is at rest, and wherein the control system is configured maintain a decreased negative pressure based on a determination that the user is moving.

3. The garment of claim 2, further comprising a valve operably coupled to the control system, wherein the valve is configured to allow air to enter the enclosed region, and wherein the control system is configured to open the valve based on a determination that the user is moving, and wherein the control system is configured to close the valve based on a determination that the user is at rest.

4. The garment of claim 1, wherein the control system is detachably coupled to at least one of the cover and the pump.

5. The garment of claim 1, wherein the control system includes a power source and an electro-mechanical pressure switch electrically coupled to the power source, and wherein the electro-mechanical pressure switch is configured to electrically couple the pump to the power source in response to the pressure exceeding a threshold value.

6. The garment of claim 1, wherein the control system is configured to maintain the pressure within the enclosed region in a range between approximately negative 105 mm Hg and negative 145 mm Hg.

7. The garment of claim 1, wherein the control system comprises a power monitoring system configured to measure an amount of current supplied to the pump, wherein the power monitoring system is configured to deactivate the pump based on a determination that the amount of current is below a threshold current value.

8. The garment of claim 1, wherein the garment further comprises a sensor configured to collect data comprising at least one of mobility data and a condition of the enclosed region, and wherein the control system further comprises a transceiver configured to transmit the data to a user device.

9. The garment of claim 8, wherein the sensor is one of a temperature sensor configured to measure a temperature of the enclosed region, a humidity sensor configured to measure a moisture level of the enclosed region, a pressure sensor configured to measure the pressure of the enclosed region, an accelerometer configured to measure movement, and a pH sensor configured to measure a pH of a user's skin.

10. The garment of claim 1, further comprising at least one of a filter configured to minimize odors from escaping the enclosed region and a filter configured to prevent ingress of fluids into the pump.

11. The garment of claim 1, wherein the cover is disposable and at least one of the pump and the control system are reuseable.

12. A system, comprising:
a power source configured to supply power to a pump; and
a sensor electrically coupled to the power source and the pump, wherein the system is configured to maintain an increased negative pressure within an enclosed region between a limb or a joint and a cover when a user is at rest, and wherein the system is configured to maintain a decreased negative pressure within the enclosed region when the user is moving.

13. The system of claim 12, wherein the sensor is configured to measure data comprising at least one of mobility data and a condition of the enclosed region, wherein the system further comprises a transceiver configured to transmit the data to a user device.

14. The system of claim 12, further comprising a processing circuit operably coupled to the pump and the sensor, wherein the sensor is configured to measure mobility data, wherein the processing circuit is configured to determine whether the user is moving or at rest based on the mobility data, wherein the processing circuit is configured to maintain an increased negative pressure based on a determination that the user is at rest, and wherein the processing circuit is configured to maintain a decreased negative pressure based on a determination that the user is moving.

15. The system of claim 14, wherein the processing circuit is configured to maintain an increased negative pressure by at least one of activating the pump, increasing an operating speed of the pump, and closing a valve.

16. The system of claim 14, wherein the processing circuit is configured to maintain a decreased negative pressure by at least one of deactivating the pump, reducing an operating speed of the pump, and opening a valve.

17. The system of claim 12, wherein the sensor comprises an electro-mechanical pressure switch, and wherein the electro-mechanical pressure switch is configured to electrically couple the pump to the power source in response to the pressure exceeding a threshold value.

18. The system of claim 12, wherein the system is configured to maintain the pressure within the enclosed region in a range between approximately negative 120 mm Hg and negative 145 mm Hg.

19. The system of claim 12, wherein the cover is disposable and at least one of the pump and the sensor are reusable.

20. The system of claim 12, further comprising:
a memory configured to store a threshold current value; and
a processing circuit operably coupled to the memory, the power source, and the pump, wherein the processing circuit is configured to monitor an amount of current supplied to the pump, and wherein the processing circuit is configured to deactivate the pump based on a determination that the amount of current is below the threshold current value.

21. The system of claim 20, wherein the memory is configured to store a threshold rate of change and a cycling frequency, wherein the processing circuit is configured to activate and deactivate the pump at the cycling frequency, wherein the processing circuit is configured to determine a rate of change of the amount of current, and wherein the processing circuit is configured to reduce the cycling frequency based on a determination that the rate of change is less than the threshold rate of change.

22. The system of claim 12, further comprising a user interface and a processing circuit operably coupled thereto, wherein the processing circuit is configured to generate an alert based on a determination that the processing circuit is separated from the pump, and wherein the user interface is configured to display the alert.

23. The system of claim 12, further comprising:
a locking member configured to prevent removal of a processing circuit from the cover; and
a transceiver configured to receive commands from a user device, wherein the processing circuit is configured to operate the locking member in response to the commands.

24. A method of making a garment comprising:
providing a cover configured to substantially surround and sealably engage at least one of a limb and a joint to form an enclosed region;
providing a pump configured to draw a negative pressure within the enclosed region;
providing a control system configured to control the pump and regulate a negative pressure within the enclosed region;
integrating the pump into the cover;
coupling the control system to at least one of the cover and the pump; and
electrically coupling the pump to the control system.

25. The method of claim 24, further comprising:
detachably coupling the control system to at least one of the cover and the pump.

26. The method of claim 24, further comprising:
providing a valve configured to allow air to enter the enclosed region; and
integrating the valve into the cover.

27. The method of claim 24, further comprising:
providing a sensor configured to activate the pump in response to the pressure exceeding a threshold value;
providing a power source;
integrating the sensor into the cover;
coupling the power source to the cover; and
electrically coupling the sensor to the pump and the power source.
